(19) United States
(12) Patent Application Publication
(10) Pub. No.: US 2024/0008922 A1
(43) Pub. Date: Jan. 11, 2024

(54) ULTRAHYDROPHOBIC LASER COATING AND METHOD

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Teo Heng Jimmy Yang, Heath (GB); Kurt G. Shelton, Bedford, MA (US)

(21) Appl. No.: 18/036,532

(22) PCT Filed: Nov. 12, 2021
(86) PCT No.: PCT/US2021/059134

§ 371 Date: May 11, 2023

Related U.S. Application Data
(60) Provisional application No. 63/113,461, filed on Nov. 13, 2020.

(51) Int. Cl. A61B 18/22

(52) U.S. Cl. CPC A61B 18/22; A61B 2018/2266; A61B 2018/00125

(57) ABSTRACT

A laser system, laser fibers, and associated methods are disclosed. In one example, the devices include a hydrophobic coating with a hydrophobic nanoscale physical structure.

### CLAIM OF PRIORITY

This patent application claims the benefit of priority to U.S. Provisional patent application Ser. No. 63/113,461, entitled “ULTRAHYDROPHOBIC LASER COATING AND METHOD,” filed on Nov. 13, 2021, which is hereby incorporated by reference herein in its entirety.

### TECHNICAL FIELD

Embodiments described herein generally relate to medical devices. Specific examples of medical devices include medical laser devices. Specific example devices include fiber optic lasers that may be introduced to tissue through a lumen.

### BACKGROUND

Several medical devices will benefit from a reduction in adhesion of material to one or more surfaces. For example, in laser devices, a reduced adhesion can prevent unwanted occlusion of optical surfaces and unwanted friction between laser fibers and lumen walls. Improved laser devices and other medical devices with reduced adhesion surfaces are desired.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a laser system in accordance with some example embodiments.

FIG. 2 shows a number of different laser fibers for use with the system shown in FIG. 1 in accordance with some example embodiments.

FIG. 3 shows a portion of a laser fiber in accordance with some example embodiments.

FIG. 4 shows another portion of a laser fiber in accordance with some example embodiments.

FIG. 5 shows a hydrophobic structure of a coating on a surface of a laser fiber in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

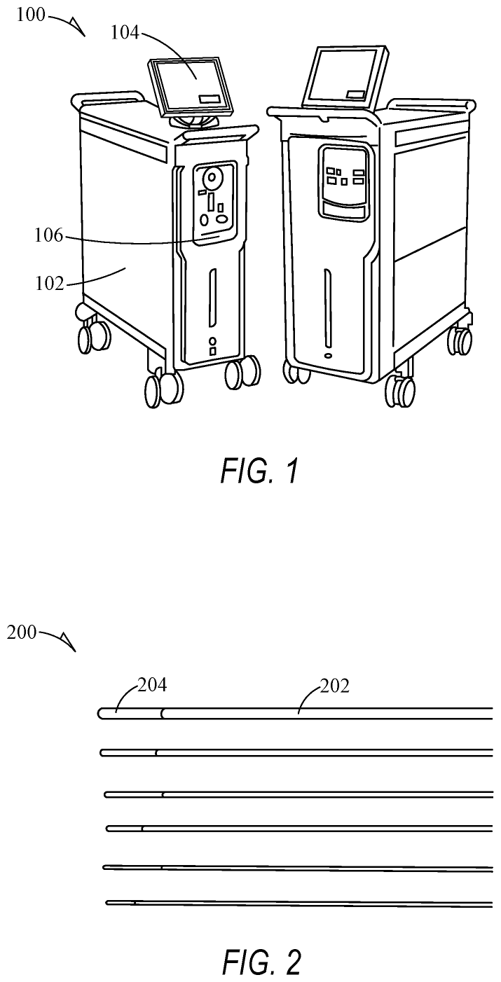

FIG. 1 shows one example of a laser system 100. In one example, the laser system includes a yttrium aluminum garnet (YAG) laser system. In one example, the laser system 100 includes a pulsed laser system. A housing 102 is shown, that includes a number of system components. Examples of system components include, but are not limited to, power supply circuitry, a lasing media, one or more pumping lamps, a cooling system, and control circuitry. In the example shown, a display screen 104, and control interface 106 are included. Although some specific components are described for example laser systems, the invention is not so limited. Other types of lasers and corresponding laser components may also be used.

FIG. 2 shows a number of examples of laser fibers 200. The fibers 200 may vary in any of a number of metrics, including, but not limited to, diameter, tip configuration, etc. The example fibers 200 of FIG. 2 show an axial portion 202 and a distal end 204 of the fibers 200.

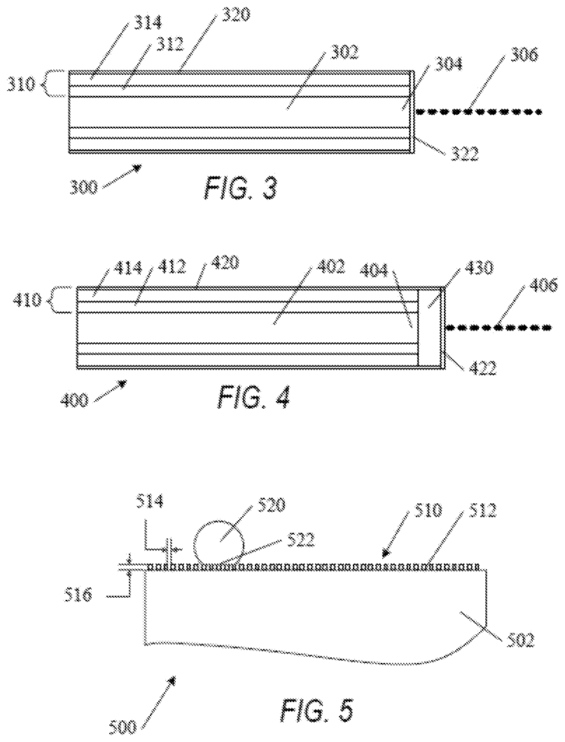

FIG. 3 shows a distal end of an example fiber 300. The fiber 300 includes a core 302 and one or more outer layers 310. In one example, the outer layers 310 include a cladding 312, a jacket 314, and a low adhesion coating 320. In one example, the core 302 includes silica. Other glass materials apart from silicon oxide are also within the scope of the invention. In one example, the cladding 312 includes a doped silica layer. Other materials with an index of refraction different than the core 302 are also within the scope of the invention. In one example, the jacket 314 includes a polymer coating. In one example, the jacket protects the cladding 312 and core 302 from mechanical damage, such as scratching.

In the example shown, the fiber 300 includes a low adhesion outer coating 320. In the example of FIG. 3, the low adhesion outer coating 320 covers all lateral surfaces of the fiber 300. In one example, the low adhesion outer coating 320 covers a portion of a lateral surface of the fiber. In one example a low adhesion outer coating 322 also coats a fiber tip 304 where laser light 306 exits the fiber 300. In one example a low adhesion outer coating 322 only coats a fiber tip 304 where laser light 306 exits the fiber 300 and coating 320 is not used. Examples of low adhesion coating 320, 322 are described in more detail below under discussion of FIG. 5.

FIG. 4 shows another example of a fiber 400. Similar to the example of FIG. 3, the fiber 400 includes a core 402 and one or more outer layers 410. In one example, the outer layers 410 include a cladding 412, a jacket 314, and a low adhesion coating 420. Example materials for fiber 400 include materials and configurations similar to fiber 300 described above.

In one example, fiber 400 includes a protective tip 430 coupled to a fiber tip 404 where laser light 406 exits the fiber 400. In one example the protective tip 430 may include a lens shaped to alter or focus the laser light 406. In one example, the protective tip 430 may be configured to not alter the laser light 406 in any perceivable or functionally significant, for example by configuring flat interfaces that allow substantially undisturbed transmission of the laser light 406. In one example the protective tip 430 may include sapphire.

In the example shown, the fiber 400 includes a low adhesion outer coating 420. In the example of FIG. 4, the low adhesion outer coating 420 covers all lateral surfaces of the fiber 400. In one example, the low adhesion outer coating 420 covers a portion of a lateral surface of the fiber. In one example a low adhesion outer coating 422 also coats the protective tip 430. In one example a low adhesion outer coating 422 only coats the protective tip 430 and coating 420 is not used.

Examples of low adhesion coating 320, 322, 420, 422 are described in more detail below under discussion of FIG. 5.

FIG. 5 shows one example of a low adhesion coating 510 on a substrate 502. As shown in examples above, the coating 510 may be on all or a portion of a surface. For example, the coating 510 may be on an entire inner surface of a lumen. The coating 510 may be on only a portion of an inner surface of a lumen. The coating 510 may be on an entire outer surface of a lumen. The coating 510 may be on only a portion of an outer surface of a lumen. The coating 510 may be on both inner and outer surfaces of a lumen. The coating 510 may be on all or a portion of a surface of an inner device.

As shown in FIG. 5, in one example, the coating 510 includes a hydrophobic physical structure with asperities 512 having a height 516 and a pitch 514. The hydrophobic structure illustrated for the representative coating 510 can be described by the following equation:

$${Λ}_{C} = \frac{- ρ {{gV}^{1 / 3} ( ( \frac{1 - cos ( {θ}_{a} )}{sin ( {θ}_{a} )} ) ( 3 + {( \frac{1 - cos ( {θ}_{a} )}{sin ( {θ}_{a} )} )}^{2} ) )}^{2 / 3}}{{( 36 π )}^{1 / 3} γ cos ( {θ}_{a , 0} + w - 90 )}$$

where Λ is a contact line density, and Λ_{c} is a critical contact line density; ρ=density of the liquid droplet; g=acceleration due to gravity; V=volume of the liquid droplet; θₐ=advancing apparent contact angle; θ_{a,0}=advancing contact angle of a smooth substrate; γ=surface tension of the liquid; and w=tower wall angle.

The contact line density Λ is defined as a total perimeter of asperities over a given unit area.

In one example, if Λ>Λ_{c} then a droplet 520 of liquid are suspended in a Cassie-Baxter state. Otherwise, the droplet 520 will collapse into a Wenzel state. In one example when a Cassie-Baxter state is formed, an ultra-hydrophobic condition exists and a low adhesion coating is formed. FIG. 5 illustrates a Cassie-Baxter state, where the droplet 520 rests on top of the asperities 512. In one example, a coating thickness 516 is between 10 and 300 nanometers.

In one example, the asperities are formed by application of nanoparticles to a surface of the substrate 502. In one example, the nanoparticles include hexamethyldisiloxane (HIVID SO) particles. In one example, the nanoparticles include tetramethyldisiloxane (TMDSO) particles. In one example, the nanoparticles include fluorosilane particles. Other nanoparticle materials are also within the scope of the invention. In one example, a hydrophobic chemistry of the nanoparticle, in combination with a nano scale asperity structure as shown in FIG. 5 provide better hydrophobicity compared to a hydrophobic chemistry alone.

In one example, application of appropriately sized nanoparticles provides the desired structure of asperities. In one example, etching creates all or a part of the desired structure of asperities. In one example, etching includes ion etching to form the desired structure of asperities.

In one example, the addition of a low adhesion coating on surfaces of laser fibers provide a number of advantages. Fibers having a low adhesion coating show reduced adhesion over other non-textured coatings for bio materials including, but not limited to, tissues, blood, fats, and/or other biological materials. Fibers having a low adhesion coating may have lower friction with an interior surface of a lumen used to introduce the fibers. Low adhesion coatings on a tip of a fiber where laser light exits may also prevent adhesion of tissue or body fluids. Additionally, due to the extremely thin nature of the low adhesion coatings described and the particular chemistries of the low adhesion coatings described, there will be little or no distortion of laser light passing through coatings such as coatings 322 and 422 described above. For example, fluorosilane coatings as described are substantially transparent, without any perceivable attenuation to laser light. Other chemistries described include similar desirable transparency and little or no attenuation.

Tips of laser fibers are a particularly difficult environment to protect from unwanted adhesion. The tip may be locally heated from the laser which encourages unwanted adhesion. Additionally, vaporization of tissue or fluids near the tip create a shock force that may damage a tip of a fiber. Examples of low adhesion coatings described are not damaged by heat from the laser, and are able to withstand the shock forces from vaporization. In particular, examples that include a protective tip coupled to a fiber tip with a low adhesion coating on an outer surface will resist unwanted adhesion and be resistant to damage from vaporization shock forces.

To better illustrate the method and apparatuses disclosed herein, a non-limiting list of embodiments is provided here:

Example 1 includes a laser system. The laser system includes a lasing medium, a pumping lamp adjacent to the lasing medium, circuitry to control the pumping lamp, and a laser fiber coupled to a lasing medium outlet. The laser fiber includes a core for transmitting laser light, and a coating on at least a portion of the laser fiber, wherein the coating includes a hydrophobic physical structure.

Example 2 includes the laser system of example 1, wherein the coating includes nanoparticles that form the hydrophobic physical structure.

Example 3 includes the laser system of any one of examples 1-2, wherein the coating includes hexamethyldisiloxane (HMDSO).

Example 4 includes the laser system of any one of examples 1-3, wherein the coating includes fluorosilane.

Example 5 includes the laser system of any one of examples 1-4, further including a polymer jacket covering lateral surfaces of the core, and wherein the coating is on the polymer jacket.

Example 6 includes the laser system of any one of examples 1-5, wherein the circuitry is configured as a pulsed laser system.

Example 7 includes a laser device. The laser device includes a laser fiber adapted for coupling to a lasing medium outlet, including a core for transmitting laser light; and a coating on at least a portion of the laser fiber, wherein the coating includes a hydrophobic physical structure.

Example 8 includes the laser device of example 7, wherein the coating includes nanoparticles that form the hydrophobic physical structure.

Example 9 includes the laser device of any one of examples 7-8, wherein the coating includes hexamethyldisiloxane (HMDSO).

Example 10 includes the laser device of any one of examples 7-9, wherein the coating includes fluorosilane.

Example 11 includes the laser device of any one of examples 7-10, further including one or more outer layers covering lateral surfaces of the core, and wherein the coating is an outermost layer of the one or more outer layers. Example 12 includes the laser device of any one of examples 7-11, wherein the coating covers an entire outer surface of the laser fiber.

Example 13 includes a laser device. The laser device includes a laser fiber adapted for coupling to a lasing medium outlet. The laser fiber includes a core for transmitting laser light, a lens at an end of the core, and a coating on the lens, wherein the coating includes a hydrophobic physical structure.

Example 14 includes the laser device of example 13, wherein the lens is flat at an orientation normal to a direction of laser light, when in operation.

Example 15 includes the laser device of any one of examples 13-14, wherein the lens is shaped to alter or focus laser light, when in operation.

Example 16 includes the laser device of any one of examples 13-15, wherein the lens includes sapphire material.

Example 17 includes the laser device of any one of examples 13-16, further including a second coating on at least a portion of a lateral surface of the laser fiber, wherein the second coating includes a hydrophobic physical structure.

Example 18 includes the laser device of any one of examples 13-17, wherein the coating includes nanoparticles that form the hydrophobic physical structure.

Example 19 includes the laser device of any one of examples 13-18, wherein the coating includes hexamethyldisiloxane (HMDSO).

Example 20 includes the laser device of any one of examples 13-19, wherein the coating includes fluorosilane.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. A laser system comprising:
a lasing medium;
a pumping lamp adjacent to the lasing medium;
circuitry to control the pumping lamp;
a laser fiber coupled to a lasing medium outlet, including:
a core for transmitting laser light; and
a coating on at least a portion of the laser fiber, wherein the coating includes a hydrophobic physical structure.

2. The laser system of claim 1, wherein the coating includes nanoparticles that form the hydrophobic physical structure.

3. The laser system of claim 1, wherein the coating includes hexamethyldisiloxane (HMDSO).

4. The laser system of claim 1, wherein the coating includes fluorosilane.

5. The laser system of claim 1, further including a polymer jacket covering lateral surfaces of the core, and wherein the coating is on the polymer jacket.

6. The laser system of claim 1, wherein the circuitry is configured as a pulsed laser system.

7. A laser device, comprising:
a laser fiber adapted for coupling to a lasing medium outlet, including:
a core for transmitting laser light; and
a coating on at least a portion of the laser fiber, wherein the coating includes a hydrophobic physical structure.

8. The laser device of claim 7, wherein the coating includes nanoparticles that form the hydrophobic physical structure.

9. The laser device of claim 7, wherein the coating includes hexamethyldisiloxane (HMDSO).

10. The laser device of claim 7, wherein the coating includes fluorosilane.

11. The laser device of claim 7, further including one or more outer layers covering lateral surfaces of the core, and wherein the coating is an outermost layer of the one or more outer layers.

12. The laser device of claim 7, wherein the coating covers an entire outer surface of the laser fiber.

13. A laser device, comprising:
a laser fiber adapted for coupling to a lasing medium outlet, including:
a core for transmitting laser light;
a lens at an end of the core; and
a coating on the lens, wherein the coating includes a hydrophobic physical structure.

14. The laser device of claim 13, wherein the lens is flat at an orientation normal to a direction of laser light, when in operation. The laser device of claim 13, wherein the lens is shaped to alter or focus laser light, when in operation.

16. The laser device of claim 13, wherein the lens includes sapphire material.

17. The laser device of claim 13, further including a second coating on at least a portion of a lateral surface of the laser fiber, wherein the second coating includes a hydrophobic physical structure.

18. The laser device of claim 13, wherein the coating includes nanoparticles that form the hydrophobic physical structure.

19. The laser device of claim 13, wherein the coating includes hexamethyldisiloxane (HMDSO).

20. The laser device of claim 13, wherein the coating includes fluorosilane.
